(19) United States
(12) Patent Application Publication
(10) Pub. No.: US 2003/0229140 A1
(43) Pub. Date: Dec. 11, 2003

(54) Herbal molecule as potential anti-leukemic drug

(76) Inventors: Santu Bandyopadhyay, Calcutta (IN); Bikash Chandra Pal, Kolkata (IN); Samir Bhattacharya, Kolkata (IN); Keshab Chandra Roy, Kolkata (IN); Gautam Bandyopadhyay, Kolkata (IN)

Correspondence Address: FITZPATRICK CELLA HARPER & SCINTO, 30 ROCKEFELLER PLAZA, NEW YORK, NY, 10112, US

(21) Appl. No.: 10/338,688

(22) Filed: Jan. 9, 2003

Related U.S. Application Data
(60) Provisional application No. 60/384,163, filed on May 31, 2002.
(60) Provisional application No. 60/393,750, filed on Jul. 8, 2002.

(30) Foreign Application Priority Data
Dec. 20, 2002 (WO) PCT/IB02/05565

(51) Int. Cl. A61K 31/235

(52) U.S. Cl. 514/533

(57) ABSTRACT

The present invention relates to a new use of the compound chlorogenic acid isolated from the piper betel leaf extract or from any other sources for the treatment of acute and chronic myeloid leukemia and lymphoid leukemia, and the present invention also provides a pharmaceutical composition comprising chloregenic acid along with pharmaceutically acceptable additive for the treatment of acute and chronic myeloid leukemia and lymphoid leukemia.

This Application claims benefit of U.S. Provisional Application No. 60/393,750 filed Jul. 8, 2002; No. 60/384,163 filed May 31, 2002; and PCT Application No. PCT/IB 02/05565 filed Dec. 20, 2002.

### TECHNICAL FIELD

This invention relates to treatment of acute and chronic myeloid leukemia (AML and CML) and also of lymphoid leukemia by Chlorogenic acid (CA) isolated from Piper betel leaf extract. Myeloid leukemia, both acute (AML) and chronic (CML) and lymphoid leukemia are lethal, there is no drug directing towards the destruction of the leukemic cells, and these cells poorly respond to chemotherapy which is always non-specific thus adversely affecting normal cells. Unique property of the therapy with Piper betel component (Chlorogenic acid) is the killing of myeloid cancer cells and lymphoid cancer cells leaving other normal cells unaffected.

### BACKGROUND AND PRIOR ART REFERENCES

Myeloid leukemia is usually subdivided into two groups: Acute Myeloid Leukemia (AML) and Chronic Myeloid Leukemia (CML). AML is characterized by an increase in the number of myeloid cells in the bone marrow and an arrest in their maturation. In the United States, the annual incidence of AML is approximately 2.4 per 100,000 and it increases progressively with age, to a peak of 12.6 per 100,000 adults 65 years of age or older. The CML is a malignant clonal disorder of hematopoietic stem cells. The median age at presentation is 53 years, but it occurs at all age groups, including children. The natural history of CML is progression from a benign chronic phase to a rapidly fatal blast crisis within three to five years or even earlier. The prognosis of CML is also poor in spite of vast advancement of clinical medicine (1). CD33 represents a specific and useful marker in the process of myeloid cell differentiation (2). Recent reports suggest that engagement of CD33 by monoclonal antibody induced apoptosis leading to growth inhibition of proliferation of AML and CML cells in vitro (2, 3). Exploiting the myeloid specific expression of CD33, humanized anti-CD33 monoclonal antibody conjugated with anti-cancer drug has been tried in AML patients with significant success (4). Similarly, lymphoid leukemia is also subdivided in two groups: acute lymphocytic leukemia (ALL) and chronic lymphocytic leukemia (CLL). Lymphoid leukemia may affect both T and B cell lineages and are prevalent in children. With the extracts from Piper betel leaves anti-myeloid activity was claimed earlier (Patent filed no. PCT/INOO/00118 dated Dec. 12, 2000) and 3-o-p-Coumaryl quinic acid, an active factor for the treatment of AML and CML on 30.05.2002 U.S. provisional patent Application No. 60/384,163 (299/NF/2002.)

Hence, applicant's earlier findings are in direct consonance with the present patent filing on Chlorogenic acid (CA) isolated from the fractions of the betel leaf extracts for treating acute and chronic myeloid leukemia and lymphoid leukemia. Chlorogenic acid is known to have anti-allergic activity (5). CA also inhibits hepatic and renal glucose-6-phosphatase systems (6). CA is an inhibitor of epidermal lypoxygenase activity and TPA-induced ear inflammation (7). CA also renders inhibitory effects on TPA-induced tumor promotion in mouse skin (7). Anti-HIV activity of CA has also been reported (8). Although inhibition of tumor promotion has been attributed to CA, no anti-tumor activity on established tumors including anti-leukemic activity has not been reported for CA. In the present patent application, anti-leukemic activity and the anti-tumor activity of CA are claimed for the first time.

Piper betel leaves have a strong pungent aromatic flavour and are widely used in India as a masticatory. Generally, mature or over mature leaves, which have ceased growing but not yet become brittle are used for chewing. The basic preparation for chewing purposes consists of betel leaf smeared with hydrated lime and catechu to which scrapings of arecanut are added; flavorings such as coconut shavings, clove, cardamom, fennel, powdered liquorice, nutmeg and also tobacco are used according to one's taste. In some places prepared Piper betel leaf preparation is covered with silver or gold film. As a masticatory, it is credited with many properties: it is aromatic, digestive, stimulant and carminative. Medicinally it is useful in catarrhal and pulmonary infections; it is also used for poultices. The effects of chewing of betel leaves with arecanut and other adjuncts are the excitation of the salivary glands and the irritation of the mucous membrane of the mouth. The red coloration produced is due to a pigment in the arecanut, which manifests itself under the action of alkali in lime and catechu. A mild degree of stimulation is produced, resulting in a sensation of warmth and well being, besides imparting a pleasant odour. The most important factor determining the aromatic value of the leaf is the amount and particularly the nature of the essential oil present. Betel leaves from different regions vary in smell and taste. The most pungent is the Sanchi type, while the most mild and sweet ones are from Varanasi.

The betal leaves contain essential oils, the content of oil varies from 0.7 to 2.6 percent depending upon the varieties of leaves. The oil consists of phenols and terpens. The higher the proportion of phenol oil, the better the quality. An isomer of eugenol named chavibetol (betel phenol; 4-alkyl-2-hydroxy-1methoxy benzene) is considered to be characteristic constituent of betel oil. Betel oil of Indian types contains as a predominant phenolic constituent and used in the treatment of various respiratory problems, either as a local application or by gargle. It has carminative properties. It exhibits in different actions on the central nervous system of mammals. The essential oil and extracts of the leaves possess activity against several Gram-positive and Gram-negative bacteria such as Micrococcus pyogenes var. Albus, Bacillus subtilis and B. Megaterium, Diplococcus pneumoniae, Streptococcus pyogenes, Escherichia coli, Salmonella typhosa, Vibrio comma, Shigella dysenteriae, Proteus vulgaris, Pdseudomonas solanacaerum, Sarcina lutea and Erwinia carotorora. The essential oil and leaf extracts also showed anti-fungal activity against Asperigillus niger and A. Oryzae, Curvularia lunata and Fusarium oxysporum. The oil is found to be lethal in about 5 minutes to the protozoa, Paramaeceum caudatum (5). Steam-distillate of the leaves showed activity against Mycobacterium tyberculosis.

### REFERENCES

1. Sawyers C L, The New England Journal of Medicine, 340 (17): 1330-1340, 1999.

2. Vitale, C; Romagnani, C, et al., Proc. Natl. Acd. Sci. USA, 96 (26): 15091-15096, 1999.

3. Vitale, C et al., Proc. Natl. Acd. Sci, USA., 98 (10): 5764-5769, 2001.

4. Sievers E L, Appelbaum, F R et al., Blood, 93: 3678-3684, 1999.

5. Ito H, Miyazaki T, Ono M and Sakurai H. Bioorg. Med. Chem. 6(7): 1051-1056, 1998.

6. Arion W J et. al. Arch. Biochem. Biophys. 351(2): 279-285, 1998.

7. Conney A H et.al. Adv. Enzyme Regul. 31: 385-396, 1991.

8. Supriyatna G et.al. Phytomedicine, 7 (Suppl. II): 87, 2000.

### OBJECTS OF THE INVENTION

The main objective of the invention is to provide a new use of the compound Chlorogenic acid isolated from the Piper betel leaf extract or from any other sources for the treatment of acute and chronic myeloid leukemia and lymphoid leukemia.

Another objective of the invention is to provide a new pharmaceutical composition comprising a carrier along with the compound Chlorogenic acid for the treatment of acute and chronic myeloid leukemia and lymphoid leukemia.

Yet another objective of the invention is to provide a process for the isolation of an active fraction from leaves or any other plant parts of Piper betel to treat AML, CML and lymphoid leukemia.

Yet another objective of the invention is to provide a simplified method of isolation of active components from all plant parts of Piper betel possessing biological activities relevant to the treatment of AML, CML and lymphoid leukemia.

Yet another objective of the invention is to provide a herbal product from leaves or any other plant parts of Piper betel for the treatment of AML, CML and lymphoid leukemia.

Yet another objective of the invention is to provide a herbal Chlorogenic acid purified from leaves of Piper betel for the treatment of AML, CML and lymphoid leukemia.

Yet another objective of the invention is to provide a process for the preparation of extract from leaves or any other plant parts of Piper betel for the treatment of AML, CML and lymphoid leukemia.

Yet another objective of the invention is to provide a simplified method of extract preparation from leaves or any other plant parts of Piper betel for the treatment of AML, CML and lymphoid leukemia.

Yet another objective of the invention is to provide a process for the preparation of Chlorogenic acid from leaves of Piper betel for the treatment of AML, CML and lymphoid leukemia.

Yet another objective of the invention is to provide herbal Chlorogenic acid purified from leaves or any other plant parts of Piper betel for the treatment of solid tumors including lymphomas.

Yet another objective of the invention is to provide new uses of Chlorogenic acid (isolated from any source or synthetically prepared) for the treatment of acute and chronic myeloid leukemia, lymphoid leukemia and solid tumors including lymphomas.

### SUMMARY OF THE INVENTION

Accordingly, the present invention provides a new use of the compound chlorogenic acid isolated from the piper betel leaf extract or from any other sources for the treatment of acute and chronic myeloid leukemia and lymphoid leukemia. In addition the present invention provides a pharmaceutical composition for acute and chronic myeloid leukemia in animals and humans, said composition comprising effective amount of chlorogenic acid (CA) and/or 3-o-p-Coumaryl quinic acid (PCQ), isolated from any plant parts of Piper betel or any other natural or synthetic source and pharmaceutical acceptable additives.

### DETAILED DESCRIPTION OF THE INVENTION

In accordance, the present invention provides a pharmaceutical composition for acute and chronic myeloid leukemia in animals and humans, said composition comprising effective amount of chlorogenic acid (CA) and/or 3-o-p-Coumaryl quinic acid (PCQ), isolated from any plant parts of Piper betel or any other natural or synthetic source and pharmaceutical acceptable additives.

One embodiment of the invention, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

Another embodiment, ratio of CA and PCQ present in the composition ranging from 1:0 to 1:10, which is effective for the treatment of solid tumors including lymphomas.

Still another embodiment, the said composition is administered through oral, intravenous, intramuscular or subcutaneous routes.

Yet another embodiment, the composition is administered at a dose level ranging between 1 and 50 mg per kg body weight/day for at least for a period of four weeks.

Yet another embodiment, the composition is administered for a period ranging between four weeks and twelve weeks.

In yet another embodiment, the growth inhibition of leukemic cell lines 2×10⁶/ml/well of cell type K562 is up to 20% at a dose level of 25 μg/ml of CA.

In yet another embodiment, the growth inhibition of leukemic cell lines 2×10⁶/ml/well of cell type K562 is up to 4% at a dose level of 25 μg/ml of PCQ.

In yet another embodiment, the growth inhibition of leukemic cell lines 2×10⁶/ml/well of cell type K562 is up to 44% at a dose level containing 25 μg/ml each of CA and PCQ.

In yet another embodiment, the growth inhibition of leukemic cell lines 2×10⁶/ml/well of cell type U937 is up to 16.67 at a dose level of 25 μg/ml of CA.

In yet another embodiment, the growth inhibition of leukemic cell lines 2×10⁶/ml/well of cell type U937 is up to 2.08% at a dose level of 25 μg/ml of PCQ.

In yet another embodiment, the growth inhibition of leukemic cell lines 2×10⁶/ml/well of cell type U937 is up to 50% at a dose level containing 25 μg/ml each of CA and PCQ.

In yet another embodiment, the growth inhibition of leukemic cell lines 2×10⁶/ml/well of cell CML leukemic cells is up to 2.38 at a dose level of 25 μg/ml of CA.

In yet another embodiment, the growth inhibition of leukemic cell lines 2×10⁶/ml/well of CML leukemic cells is up to 2.38% at a dose level of 25 μg/ml of PCQ.

In yet another embodiment, the growth inhibition of leukemic cell lines 2×10⁶/ml/well of CML leukemic cells is up to 20.3% at a dose level containing 25 μg/ml each of CA and PCQ.

One more embodiment provides the use of composition for the treatment of acute and chronic myeloid leukemia (AML & CML) and lymphoid leukemia by administering a pharmaceutical composition comprising effective amount of chlorogenic acid (CA) and/or 3-o-p-Coumaryl quinic acid (PCQ), isolated from any plant parts of Piper betel or any other source and/or in combination with pharmaceutically acceptable additives.

The invention is described herein below with reference to examples, which are illustrative only and should not be construed to limit the scope of the present invention in any manner.

### BRIEF DESCRIPTION OF THE ACCOMPANYING DRAWINGS

FIG. 1 represents HPLC of Fraction E showing the retention time (RE) of different peaks be divided into three zones, Z_{A}, Z_{B} and Z_{C}.

FIG. 1a. The peaks have been numbered (Z_{A had} 4 major peaks whereas Z_{B} had very small to moderate 10 peaks and Z_{C} had two distinct peaks)

FIG. 3 represents the structure of Chlorogenic acid (CA) or 3-Caffeoyl quinic acid.

FIG. 4 represents photomicrographs of Leukemic cell lines, CML Patient's and normal donor's PBMC after treatment with CA.

### EXAMPLE-1

Collection of Plant Material

The leaves and all other plant parts of Piper betel were collected from the climber from different areas and West Bengal, India. A voucher specimen was deposited at the Deptt. of Medicinal Chemistry at the Indian Inst. of Chemical Biology, 4 Raja S.C. Mullick Road, Kolkata-700 032.

### EXAMPLE 2

### Isolation of Compound Chlorogenic Acid

4.7 Kg of Piper betel leaves freshly collected, washed with distilled water and then cut into small pieces. Small pieces of leaves were gathered together, mixed with 1.0 litre of distilled water, and thoroughly homogenized in a mixture blender. The homogenate was passed through a fine cheesecloth to filter out the large particles and the filtrate was collected. The process was repeated 2-3 times to have maximum yield. The combined filtrate was then centrifuged, the aliquot, a clear solution, was collected and lyophilised to a semi-solid mass, which was about 110 gm.

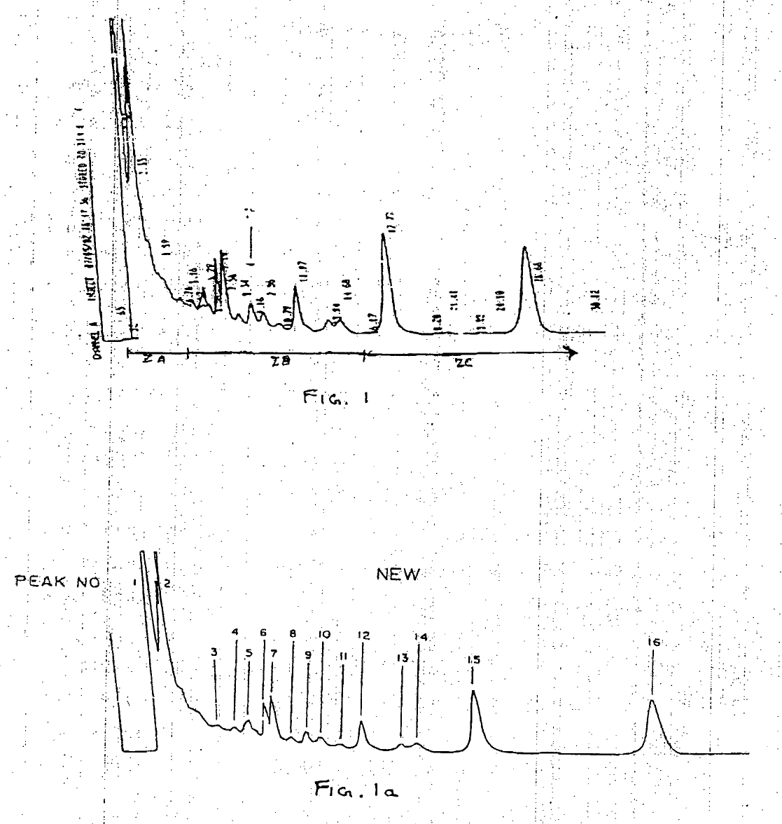

Collected material was examined for biological activity i.e. destruction of leukemic cells. On observing its positive activity, purification was initiated. 10 gm of above-mentioned material was loaded on Sephadex LH-20 column and chromatographed with water, water-methanol (1:1) and methanol as eluent. Three different fractions thus obtained from three different solvent systems were separately checked for biological activity. The activity was located only on Fraction 2 i.e. Methanol-water (1:1) and termed as fraction E. Fraction E was then subjected to HPLC analysis using Intersil ODS-3 analytical column. The column was equilibrated with methanol-water-acetic acid (23:76:1), flow rate was maintained at 1.0 ml per min and peaks were identified at 280 nm. According to the retention time (RE) different peaks could be divided into three zones, Z_{A}/Z_{B} and Z_{C} (FIG. 1). Z_{A} had 4 major peaks whereas Z_{B} had very small to moderate 10 peaks and Z_{C} had two distinct peaks. It was convenient to observe biological activity of three zones A_{A}, Z_{B} and Z_{C}, which were separately tested on leukemia cells for destructive activity. All three showed biological activity.

It may be noted here that Z_{C} which had two distinct peaks with retention time (Rₜ) 17.77 and 26.66 min. Rₜ 17.77 min peak demonstrated considerable activity and therefore its structure was determined and filed for patent (299/NF/2002; dated-30.05.2002). The latter i.e. Rₜ 26.66 min peak did not show any activity.

The Zone Z_{B} contained 10 peaks (1 to 10) of varied sizes. Pooled 1-10 peaks fraction demonstrated leukemic cell killing activity. For this reason all different peaks were separately monitored for biological activity. Major peaks amongst them were 2, 3, 4 and 8 corresponding to Rₜ 6.58, 7.14, 7.56 and 11.97 respectively and none of them exhibited activity. Hence minor peaks were separately examined by collecting repeated fractions for each peaks to increase the amount of active molecules present there. On searching for activity of these peaks i.e. 1, 5, 6, 7, 9 and 10 corresponding to Rₜ 5.16, 8.34, 9.16, 9.86, 13.94 and 14.68 respectively. Except peak no 6 (Rₜ 9.16 min) no other peaks had shown detectable activity. Peak 6 (Rₜ 9.16 min) pooled fractions were lyophilized and reran through HPLC, the sharp single peak indicate its purity (FIG. 2). This was then subjected to IR, NMR and Mass spectral analysis to determine the structure.

| | | |
|---|---|---|
| | KBr | |
| IR | γₘₐₓ cm⁻¹ | 3355(OH), 1689(CO), 1637, 1604 |
| | | 1522, 1443, 1286, 1189, 1121, 1082 |
| | | 1039, 975 and 854 |

| | |
|---|---|
| ¹H-NMŔ (CD₃OD) | 7.57 (1 H), 7.06 (1 H), 6.96 (1 H), 6.79 (1 H) |
| | 6.27 (1 H), 5.34 (1 H), 4.17 (1 H), 3.73 (1 H) |
| | and 2.13 (4 H) |
| ¹³C NMR (CD₃OD) | 176.00, 167.65, 148.56, 146.08, 145.79, 126.78, |
| | 121.98, 115.46, 114.24, 75.11, 72.45, 70.96, 70.26, |
| | 37.75 and 37.19 |
| FABMS m/Z | 355 (M⁺ + H) and 377 (M⁺ + Na) |

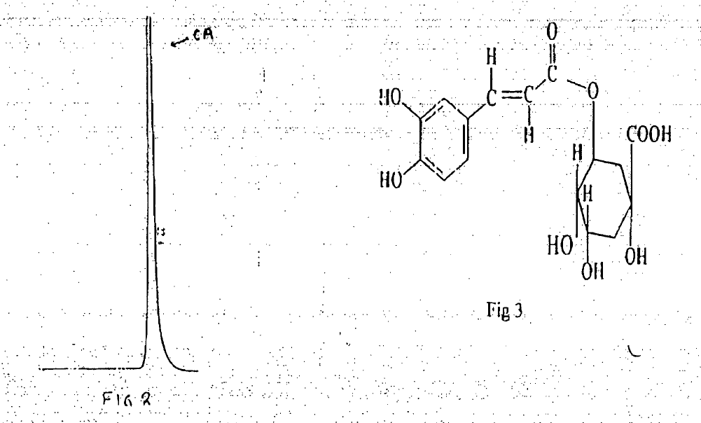

The structure thus determined shows it to be chlorogenic acid or 3-Caffeoyl quinic acid, m.p. 205°-206° C. [α]_{D}−33.25 (H₂O) (FIG. 3). The chlorogenic acid is available in the market in pure form. This was bought and compared with the chlorogenic acid isolated from Piper betle leaves, which clearly confirmed that, the available material as chlorogenic acid.

Structure filed for patent earlier was 3-p-coumaryl quinic acid which showed CD33+myeloid cells destruction but not CD33−cells, and this is very mimilar to chlorogenic acid. Except the additional hydroxyl group at C-3 position of the aromatic ring in chlorogenic acid other structures are precisely similar. Therefore, the additional activity of chlorogenic acid over the 3-p-coumaryl quinic acid can be suggested to be due to the presence of hydroxyl group at C-3 position of aromatic ring. This specific difference has given broader and stronger activity to Chlorogenic acid in destroying both CD33− and CD33+cells and also lymphoid leukemic cells.

Zone Z_{A} demonstrated bilogical activity. It contained 4 peaks, 1, 2, 3 and 4 corresponding to Rₜ 0.65, 1.32, 1.8 and 2.55 respectively. Since separation of individual peak under Z_{A} becomes difficult, location of peak wise activity could not be determined.

### EXAMPLE 3

Preparation of peripheral blood mononuclear cells (PBMC) from myeloid leukemia patients.

Whole blood (10 ml each) was drawn from previously diagnosed myeloid leukemic patients, one patient was diagnosed as AML and the other one was diagnosed as CML. Mononuclear cells were separated by Ficoll/hypaque density gradient centrifugation.

### EXAMPLE 4

Culture of Erythroleukemic cell line K562. This cell line was collected from American Type Culture Collection (ATCC), VA, USA. This cell line is CD33. K562 cells were grown in vitro in Medium RPMI-1640 containing 10% heat inactivated fetal calf serum.

### EXAMPLE 5

Culture of Promonocytic cell line U937. This cell line was obtained from American Type Culture Collection (ATCC), VA, USA, and grown in vitro as described for the cell line K562. This cell line is CD33⁺.

### EXAMPLE 6

Incubation of PBMC of myeloid leukemia patients with Chlorogenic acid (CA) in vitro.

PBMC (1×10⁵-2×10⁶/ml) of myeloid leukemia patients were incubated with varied concentrations of CA for 48 hours at 37° C. in 5% CO₂. Cells were then washed and counted for viability.

### EXAMPLE 7

Incubation of CD33⁺ cell line U937 with CA. U937 cells (1×10⁵-2×10⁶/ml) were incubated with varying concentrations of CA for 48 hours at 37° C. in 5% CO₂. Cells were then washed and counted for viability.

### EXAMPLE 8

Incubation of CD33⁻ cell line K562 with CA. K562 cells (1×10⁵-2×10⁶/ml) were incubated with varying concentrations of CA for 48 hours at 37° C. in 5% CO₂. Cells were then washed and counted for viability.

### EXAMPLE 9

Culture of T-Lymphoid cell line Molt-4. This cell line was obtained from American Type culture collection (ATCC), VA, USA and grown in vitro as described for the cell line K562.

### EXAMPLE 10

Preparation of peripheral blood mononuclear cells (PBMC) from normal individuals. Whole blood was collected and mononuclear cells were separated by Ficoll/Hypaque density gradient centrifugation.

### EXAMPLE 11

Analysis of cell cycle progression and apoptosis by Flow cytometry PBMC of a CML patient was cultured in RPMI-1640 medium supplemented with 10% heat-activated fetal bovine serum in the presence or absence of CA (100.0 μg/ml) for 48 hr. at 37° C. in 5% CO₂. Cells were fixed with 40% ethanol treated with 500 μg/ml Rnase A, and then with 69 μM propidium iodide for analysis of DNA content by Flow Cytometry as described (Mitra et al., Molecular Medicine, 6:527-541, 2000).

Results of Examples 6, 7 & 8:

As shown in Table 1, PBMC of myeloid leukemia patients, CD33⁺ Promonocytic cell line U937 and CD33⁻ Erythroleukemic cell line K562 were destroyed by Chlorogenic acid.

Results of Example 9 and 10:

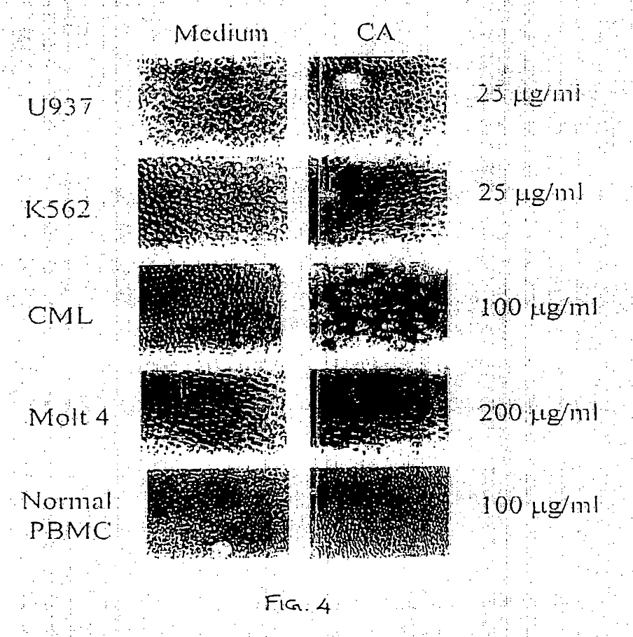

As shown Table 2, Erythroleukemia cell line K562, Pro-monocyte cell line U937 and CML patient's leukemic cells are destroyed by Chlorogenic acid T-lymphoid cell line Molt-4 requires higher dose. In contrast, PBMC of normal donors are virtually unaffected by CA. Photomicrographs of U937, K562, CML Patient's PBMC, Molt-4 and normal PBMC after culturing for 48 hr. in the presence or absence of CA are shown in FIG. 4. We claimed earlier that 3-o-p-Coumaryl quinic acid (PCQ) has anti myeloid leukemia acitivity (299/NF/2002; 30.05.2002). Here we show that combination of chlorogenic acid (CA) and 3-o-p-coumaryl quinic acid at a ratio of 1:1 is more effective than CA or PCQ alone in destroying Leukemic cell lines or Myeloid Leukemia patient's Lekemic cells but have no effects on normal donor's PBMC (Table 3).

Results of Example 11

Cell-cycle analysis demonstrate that after two days of culture, chlorogenic acid at a concentration of 100.0 μg/ml caused apoptosis in CML patients Leukemic cells in vitro because of their accumulation in S, G₂ or M phase compared to the cells cultured in medium alone (Table 4).

TABLE I
Chlorogenic acid destroys patients' leukemic cells and leukemia cell
lines in vitro.

| | Percent inhibition of cell growth after 48 hr of incubation | | | |
|---|---|---|---|---|
| | AML | CML | | |
| | patient's | patient's | Promonocytic | Erythroleukemic |
| Compound | PBMC* | PBMC | cell line (U937) | cell line (K562) |
| Medium | 0.0 | 0.0 | 0.0 | 0.0 |
| alone | | | | |
| Chlorogenic | | | | |
| acid (CA) | | | | |
| 10 μg/ml | ND | ND | 25.0 | 46.6 |
| 15 μg/ml | ND | ND | 43.7 | 93.3 |
| 20 μg/ml | 9.5 | 5.8 | 90.6 | 100.0 |
| 50 μg/ml | 16.6 | 10.3 | — | — |
| 100 μg/ml | 73.8 | 22.5 | — | — |
| 500 μg/ml | 85.7 | 32.3 | — | — |

Note:
1. PBMC*, Peripheral blood mononuclear cells.
2. The number of cells used was 1 × 10⁵/ml/well

TABLE 2
Growth Inhibition of Leukemic Cell Lines and CML Patient's
Leukemic Cells by CA in vitro.

| | Incubated | Percentage inhibition of Cell Growth | | |
|---|---|---|---|---|
| Cells | with (μg/ml) | 1 day | 2 days | 3 days |
| K562 | Medium | 0.00 | 0.00 | 0.00 |
| | CA (10.0) | 8.00 | 48.14 | 85.18 |
| | CA (25.0) | 20.00 | 62.96 | 92.59 |
| | CA (50.0) | 28.00 | 77.78 | 100.0 |
| | CA (100.0) | 84.00 | 98.14 | 100.0 |
| U937 | Medium | 0.00 | 0.00 | 0.00 |
| | CA (10.0) | 8.33 | 44.0 | 57.14 |
| | CA (25.0) | 16.66 | 52.00 | 71.42 |
| | CA (50.0 | 25.00 | 64.00 | 89.28 |
| | CA (100.0) | 45.83 | 96.00 | 100.0 |
| Molt 4 | Medium | 0.00 | 0.00 | 0.00 |
| (T lymphoid | CA (50.0) | 3.57 | 6.67 | 12.5 |
| Cell line) | CA (100.0) | 6.89 | 14.67 | 18.75 |
| | CA (200.0) | 13.79 | 46.67 | 62.5 |
| CML | Medium | 0.00 | 4.76 | 14.28 |
| Patient's | CA (50.0) | 4.76 | 22.22 | 30.0 |
| PBMC | CA (100.0 | 9.52 | 45.0 | 55.56 |
| Normal | Medium | 0.00 | 3.33 | 3.33 |
| PBMC | CA (50.0) | 6.66 | 10.34 | 15.2 |
| | CA (100.0) | 6.66 | 17.24 | 19.5 |

Of note, the number of cells used was 1 × 10⁵ per ml per well.

TABLE 3
Combination of Chlorogenic acid (CA) and 3-o-p-Coumaryl Quinic
acid (PCQ) is more effective in destroying Leukemic cell lines
and AML and CML patient's Leukemic cells in vitro

| | | % Inhibition of cell |
|---|---|---|
| | | growth (After 3 days |
| Cells | Incubated with | of incubation) |
| K562 | Media | 0.00 |
| Erythroleukemic | CA (25.0 μg/ml) | 20.00 |
| cell line | CA (50.0 μg/ml) | 28.00 |
| | PCQ (25.0 μg/ml) | 4.00 |
| | PCQ (50.0 μg/ml) | 8.00 |
| | CA (25.0 μg/ml) + PCQ (25.0 | 44.00 |
| | μg/ml) | |
| U937 | Media | 0.00 |
| Promonocyte | CA (25.0 μg/ml) | 16.67 |
| cell line | CA (50.0 μg/ml)\grave{ } | 25.00 |
| | PCQ (25.0 μg/ml) | 2.08 |
| | PCQ (50.0 μg/ml) | 4.17 |
| | CA (25.0 μg/ml) + PCQ (25.0 | 50.00 |
| | μg/ml) | |
| CML Patient's | Media | 0.00 |
| Leukemic cells | CA (25.0 μg/ml) | 2.38 |
| | CA (50.0 μg/ml)\grave{ } | 4.67 |
| | PCQ (25.0 μg/ml) | 2.38 |
| | PCQ (50.0 μg/ml) | 4.76 |
| | CA (25.0 μg/ml) + PCQ (25.0 | 20.3 |
| | μg/ml) | |
| Normal | Media | 0.00 |
| individual's | CA (25.0 μg/ml) | 1.03 |
| PBMC | CA (50.0 μg/ml)\grave{ } | 1.03 |
| | PCQ (25.0 μg/ml) | 0.00 |
| | PCQ (50.0 μg/ml) | 1.67 |
| | CA (25.0 μg/ml) + PCQ (25.0 | 1.03 |
| | μg/ml) | |

The number of cells used was 2×10⁵ per ml per well. The percentage of killing of cells by these compounds is dependent on the number of cells present per ml.

TABLE 4
The percentage of cells in different phases of cell cycle
after treatment with CA for 2 days

| | | Sub G0/G1 | | |
|---|---|---|---|---|
| | Incubated | (apoptotic 2n | G0/G1 | 2 + G2 + M |
| Type of cells | with | DNA) | (2n DNA) | (>2n DNA) |
| CML | Media | 4.65 | 51.56 | 43.87 |
| Patient's | CA (100.0 | 10.64 | 33.13 | 56.35 |
| Leukemic | μg/ml) | | | |
| Cells | | | | |

1. A pharmaceutical composition for acute and chronic myeloid leukemia in animals and humans, said composition comprising effective amount of chlorogenic acid (CA) and/or 3-o-p-Coumaryl quinic acid (PCQ), isolated from any plant parts of Piper betel or any other natural or synthetic source and pharmaceutical acceptable additives.

2. The composition as claimed in claim 1 wherein, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

3. The composition as claimed in claim 1 wherein ratio of CA and PCQ present in the composition ranging from 1:0 to 1:10, which is effective for the treatment of solid tumors including lymphomas.

4. The composition as claimed in claim 1, the said composition is administered through oral, intravenous, intramuscular or subcutaneous routes.

5. The composition as claimed in claim 1 is administered at a dose level ranging between 1 and 50 mg per kg body weight/day for at least for a period of four weeks.

6. The composition as claimed in claim 1, which is administered for a period ranging between four weeks and twelve weeks.

7. The composition as claimed in claim 1 wherein, the growth inhibition of leukemic cell lines 2×10⁶/ml/well of cell type K562 is up to 20% at a dose level of 25 μg/ml of CA.

8. The composition as claimed in claim 1, wherein the growth inhibition of leukemic cell lines 2×10⁶/ml/well of cell type K562 is up to 4% at a dose level of 25 μg/ml of PCQ.

9. The composition as claimed in claim 1, wherein the growth inhibition of leukemic cell lines 2×10⁶/ml/well of cell type K562 is up to 44% at a dose level containing 25 μg/ml each of CA and PCQ.

10. The composition as claimed in claim 1 wherein, the growth inhibition of leukemic cell lines 2×10⁶/ml/well of cell type U937 is up to 16.67 at a dose level of 25 μg/ml of CA.

11. The composition as claimed in claim 1, wherein the growth inhibition of leukemic cell lines 2×10⁶/ml/well of cell type U937 is up to 2.08% at a dose level of 25 μg/ml of PCQ.

12. The composition as claimed in claim 1, wherein the growth inhibition of leukemic cell lines 2×10⁶/ml/well of cell type U937 is up to 50% at a dose level containing 25 μg/ml each of CA and PCQ.

13. The composition as claimed in claim 1 wherein, the growth inhibition of leukemic cell lines 2×10⁶/ml/well of cell CML leukemic cells is up to 2.38 at a dose level of 25 μg/ml of CA.

14. The composition as claimed in claim 1, wherein the growth inhibition of leukemic cell lines 2×10⁶/ml/well of CML leukemic cells is up to 2.38% at a dose level of 25 μg/ml of PCQ.

15. The composition as claimed in claim 1, wherein the growth inhibition of leukemic cell lines 2×10⁶/ml/well of CML leukemic cells is up to 20.3% at a dose level containing 25 μg/ml each of CA and PCQ.

16. Use of composition as claimed in claim 1, for the treatment of acute and chronic myeloid leukemia (AML & CML) and lymphoid leukemia by administering a pharmaceutical composition comprising effective amount of chlorogenic acid (CA) and/or 3-o-p-Coumaryl quinic acid (PCQ), isolated from any plant parts of Piper betel or any other source and/or in combination with pharmaceutically acceptable additives.

17. A use as claimed in claim 16 wherein, CA and PCQ both are isolated from any plant parts of Piper betel or are synthetically prepared.

18. A use as claimed in claim 16 wherein the subject is selected from a mammal preferably a human being.

19. A use as claimed in claim 16 wherein, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

20. A use as claimed in claim 16 wherein ratio of CA and PCQ present in the composition ranging from 1:0 to 1:10, is effective for the treatment of solid tumors including lymphomas.

21. A use as claimed in claim 16 wherein, the said composition is administered through oral, intravenous, intramuscular or subcutaneous routes.

22. A use as claimed in claim 16 is administered at dose levels between 1 to 50 mg per kg body weight at least once in a day for a period of four weeks.

23. A use as claimed in claim 16, which is administered for a period of four weeks and up to three months.

24. A use as claimed in claim 16 wherein, the growth inhibition of leukemic cell lines 2×10⁶/ml/well of cell type K562 is up to 20% at a dose level of 25 μg/ml of CA.

25. A use as claimed in claim 16, wherein the growth inhibition of leukemic cell lines 2×10⁶/ml/well of cell type K562 is up to 4% at a dose level of 25 μg/ml of PCQ.

26. A use as claimed in claim 16, wherein the growth inhibition of leukemic cell lines 2×10⁶/ml/well of cell type K562 is up to 44% at a dose level containing 25 μg/ml each of CA and PCQ.

27. A use as claimed in claim 16, wherein, the growth inhibition of leukemic cell lines 2×10⁶/ml/well of cell type U937 is up to 16.67 at a dose level of 25 μg/ml of CA.

28. A use as claimed in claim 16, wherein the growth inhibition of leukemic cell lines 2×10⁶/ml/well of cell type U937 is up to 2.08% at a dose level of 25 μg/ml of PCQ.

29. A use as claimed in claim 16, wherein the growth inhibition of leukemic cell lines 2×10⁶/ml/well of cell type U937 is up to 50% at a dose level containing 25 μg/ml each of CA and PCQ.

30. A use as claimed in claim 16, wherein the growth inhibition of leukemic cell lines 2×10⁶/ml/well of cell CML leukemic cells is up to 2.38 at a dose level of 25 μg/ml of CA.

31. A use as claimed in claim 16, wherein the growth inhibition of leukemic cell lines 2×10⁶/ml/well of CML leukemic cells is up to 2.38% at a dose level of 25 μg/ml of PCQ.

32. A use as claimed in claim 17, wherein the growth inhibition of leukemic cell lines 2×10⁶/ml/well of CML leukemic cells is up to 20.3% at a dose level containing 25 μg/ml each of CA and PCQ.

33. A method of treating acute and chronic myeloid leukemia (AML & CML) and lymphoid leukemia, said method comprising administering a pharmaceutical composition comprising effective amount of chlorogenic acid (CA) and/or 3-o-p-Coumaryl quinic acid (PCQ), isolated from any plant parts of Piper betel or any other source and/or in combination with pharmaceutically acceptable additives.

34. A method as claimed in claim 33, wherein, CA and PCQ both are isolated from any plant parts of Piper betel or are synthetically prepared.

35. A method as claimed in claim 33, wherein the subject is selected from a mammal preferably a human being.

36. A method as claimed in claim 33, wherein, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

37. A method as claimed in claim 33, wherein ratio of CA and PCQ present in the composition ranging from 1:0 to 1:10, is effective for the treatment of solid tumors including lymphomas.

38. A method as claimed in claim 33, wherein the said composition is administered through oral, intravenous, intramuscular or subcutaneous routes.

39. A method as claimed in claim 33, wherein said composition is administered at dose levels between 1 to 50 mg per kg body weight at least once in a day for a period of four weeks.

40. A method as claimed in claim 33, which is administered for a period of four weeks and up to three months.

41. A method as claimed in claim 33, wherein the growth inhibition of leukemic cell lines 2×10⁶/ml/well of cell type K562 is up to 20% at a dose level of 25 μg/ml of CA.

42. A method as claimed in claim 33, wherein the growth inhibition of leukemic cell lines 2×10⁶/ml/well of cell type K562 is up to 4% at a dose level of 25 μg/ml of PCQ.

43. A method as claimed in claim 33, wherein the growth inhibition of leukemic cell lines 2×10⁶/ml/well of cell type K562 is up to 44% at a dose level containing 25 μg/ml each of CA and PCQ.

44. A method as claimed in claim 33, wherein the growth inhibition of leukemic cell lines 2×10⁶/ml/well of cell type U937 is up to 16.67 at a dose level of 25 μg/ml of CA.

45. A method as claimed in claim 33, wherein the growth inhibition of leukemic cell lines 2×10⁶/ml/well of cell type U937 is up to 2.08% at a dose level of 25 μg/ml of PCQ.

46. A method as claimed in claim 33, wherein the growth inhibition of leukemic cell lines 2×10⁶/ml/well of cell type U937 is up to 50% at a dose level containing 25 μg/ml each of CA and PCQ.

47. A method as claimed in claim 33, wherein, the growth inhibition of leukemic cell lines 2×10⁶/ml/well of cell CML leukemic cells is up to 2.38 at a dose level of 25 μg/ml of CA.

48. A method as claimed in claim 33, wherein the growth inhibition of leukemic cell lines 2×10⁶/ml/well of CML leukemic cells is up to 2.38% at a dose level of 25 μg/ml of PCQ.

49. A method as claimed in claim 33, wherein the growth inhibition of leukemic cell lines 2×10⁶/ml/well of CML leukemic cells is up to 20.3% at a dose level containing 25 μg/ml each of CA and PCQ.
